SEQUENCE LISTING (21 sequences)

SEQ ID NO: 1 - DNA, 21 bp, Artificial Sequence
  oligonucleotide
agtccctgccctttgtacaca

SEQ ID NO: 2 - DNA, 21 bp, Artificial Sequence
  oligonucleotide
gatccgagggcctcactaaac

SEQ ID NO: 3 - DNA, 20 bp, Artificial Sequence
  oligonucleotide
ggtggagagcaccaagacag

SEQ ID NO: 4 - DNA, 20 bp, Artificial Sequence
  oligonucleotide
gccggaagtcgacaatgatg

SEQ ID NO: 5 - DNA, 19 bp, Artificial Sequence
  oligonucleotide
acttccggagcgatcagtt

SEQ ID NO: 6 - DNA, 20 bp, Artificial Sequence
  oligonucleotide
cagctccttggttcttccat

SEQ ID NO: 7 - DNA, 20 bp, Artificial Sequence
  oligonucleotide
tggaagcttgtctccagtga

SEQ ID NO: 8 - DNA, 20 bp, Artificial Sequence
  oligonucleotide
cttgtggaagtcacgccttt

SEQ ID NO: 9 - DNA, 18 bp, Artificial Sequence
  oligonucleotide
gcatggtgccttcgctga

SEQ ID NO: 10 - DNA, 22 bp, Artificial Sequence
  oligonucleotide
tggcatctctgtgtcaaccatg

SEQ ID NO: 11 - DNA, 20 bp, Artificial Sequence
  oligonucleotide
agaagatcccagggaggaaa

SEQ ID NO: 12 - DNA, 20 bp, Artificial Sequence
  oligonucleotide
tcattggctatctgcagcac

SEQ ID NO: 13 - DNA, 20 bp, Artificial Sequence
  oligonucleotide
ttggagagagagcgtccaat

SEQ ID NO: 14 - DNA, 20 bp, Artificial Sequence
  oligonucleotide
ctcaaagaaggccacaaagc

SEQ ID NO: 15 - DNA, 20 bp, Artificial Sequence
  oligonucleotide
accctggtagccctgtcttt

SEQ ID NO: 16 - DNA, 20 bp, Artificial Sequence
  oligonucleotide
actcattacacgggctggtc

SEQ ID NO: 17 - DNA, 19 bp, Artificial Sequence
  oligonucleotide
ctgctgacccacaagcagt

SEQ ID NO: 18 - DNA, 21 bp, Artificial Sequence
  oligonucleotide
ggcaagatttctgtccctctc

SEQ ID NO: 19 - DNA, 26 bp, mus musculus
cctgttgctcaaccttgcacagttcc

SEQ ID NO: 20 - DNA, 26 bp, rattus norvegicus
ccttttgctcaaccttgcacagttcc

SEQ ID NO: 21 - DNA, 26 bp, homo sapiens
tgtgttgcccaatcctgaccaggtgc

(19) United States
(12) Patent Application Publication
(10) Pub. No.: US 2010/0247551 A1
(43) Pub. Date: Sep. 30, 2010

(54) Use of Lipocalin 2 in the Regulation of Insulin Sensitivity

(76) Inventor: Evan D. Rosen, Arlington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/438,632

(22) PCT Filed: Aug. 29, 2007
(86) PCT No.: PCT/US07/18992

§ 371 Date: Apr. 14, 2009

Related U.S. Application Data
(60) Provisional application No. 60/842,587, filed on Sep. 5, 2006.

(51) Int. Cl. A61K 31/07; C12Q 1/02; C12Q 1/68; G01N 33/53; A61K 39/395; C07C 43/02; C07K 16/00; A61P 3/10

(52) U.S. Cl. 424/172.1; 435/29; 435/6; 514/725; 435/7.8; 568/668; 530/389.8

(57) ABSTRACT

Methods of identifying compounds that modulate lipocalin 2 activity or expression are described, as are methods of reducing insulin resistance or increasing insulin sensitivity by administering compounds that modulate lipocalin 2 expression. Methods of diagnosing insulin resistance or related conditions, by measuring lipocalin 2 activity, are also described.

### RELATED APPLICATION

This application claims the benefit of U.S. Provisional Application No. 60/842,587, filed on Sep. 5, 2006. The entire teachings of the above application are incorporated herein by reference.

### GOVERNMENT SUPPORT

The invention was supported, in whole or in part, by a grants DK63906 and DK43051 from the National Institute of Health. The Government has certain rights in the invention.

### BACKGROUND OF THE INVENTION

The worldwide epidemic of obesity and type 2 diabetes has focused attention on adipocyte biology and the role of adipose tissue in the integration of systemic metabolism (Kahn, B. B., and Flier, J. S. (2000) J Clin Invest 106(4), 473-481). The discovery of leptin more than a decade ago established a paradigm in which secreted proteins from adipocytes coordinate energy balance and glucose homeostasis. (Halaas, J. L., et al., (1995) Science 269(5223), 543-546) Since that initial discovery, the number of adipocyte-derived signaling molecules has grown ever larger, and the term cadipokine was coined to reflect the fact that many of these molecules exert positive or negative actions on inflammation. Several adipokines promote insulin sensitivity, including leptin (Halaas, J. L., et al., (1995) Science 269(5223), 543-546); adiponectin (Scherer, P. E., et al., (1995) J Biol Chem 270(45), 26746-26749); and visfatin (Fukuhara, A., et al., (2005) Science 307(5708), 426-430), while others induce insulin resistance, such as resistin (Steppan, C. M., et al. (2001) Nature 409 (6818), 307-312); and retinol binding protein 4 (RBP4) (Yang, Q., et al., (2005) Nature 436(7049), 356-362).

Insulin resistance in the peripheral tissues such as muscle and fat is associated with increased secretion of insulin by pancreatic β-cellS. The secreted insulin promotes glucose utilization and inhibits production of glucose by the liver. However, the pancreatic β-cells often cannot sustain the increased production of insulin resulting in the eventual decrease of insulin production and glucose intolerance.

Insulin resistance is characterized, for example, by increased glucose concentration in the blood, increased insulin concentration in the blood, decreased ability to metabolize glucose in response to insulin, or a combination of any of the above. Insulin resistance is thought to predict possible later development of diabetic disease, such as Type 2 Diabetes. However, even in the absence of diabetes, insulin resistance is a major risk factor for cardiovascular disease (Despres, et al., N Engl. J. Med 334:952-957 (1996)). The loss of insulin production in insulin resistance and diabetes results in increased blood glucose or hyperglycemia. Hyperglycemia in turn can contribute to long term illness such as nephropathy, neuropathy, and retinopathy.

Insulin resistance is also associated with abnormalities in glucose and lipid metabolism, obesity, kidney disease, high blood pressure and increased risk for cardiovascular disease. The association of insulin resistance with these other abnormalities is referred to as “Insulin Resistance Syndrome” or “Metabolic Syndrome” or “Syndrome X”. In particular, Metabolic Syndrome has been characterized as the co-occurrence of obesity (especially central obesity), dyslipidemia (especially high levels of triglycerides and low levels of high density lipoprotein cholesterol), hyperglycemia and hypertension. People with Metabolic Syndrome are at increased risk for diabetes or cardiovascular disease relative to people without the syndrome (Meigs, J. B., BMJ: 327, 61-62, (2003)).

Due to the association of insulin resistance with later development of diabetes and cardiovascular disease, and the prevalence of insulin resistance worldwide, the need exists for additional metabolic or endocrine targets for the development of treatments that alleviate or mitigate diseases associated with insulin resistance. A need also exists for additional detection/diagnostic methods of insulin resistance, Metabolic Syndrome and Type II diabetes to allow for the earliest possible intervention through life-style changes and/or medication.

### SUMMARY OF THE INVENTION

The present invention provides important targets and screening methods for the identification of molecules or compounds that can be used for the development of treatments and medicaments that alleviate or mitigate symptoms and diseases associated With insulin resistance. As described herein, the invention relates to methods for identifying compounds that modulate Lcn2 activity or expression. The methods comprise contacting a test sample comprising Lcn2 (e.g., a test sample comprising cells) with a test compound and comparing the level of Lcn2 activity or expression in the presence of the test compound to the level of Lcn2 activity or expression in the absence of the test compound to determine modulation of Lcn2 activity, wherein an alteration of Lcn2 activity is indicative of a compound that modulates Lcn2 activity or expression.

The present invention also relates to methods of reducing insulin resistance or increasing insulin sensitivity in a mammal. The method comprises administering to a mammal a compound that reduces the activity or expression of Lcn2. The method additionally relates to methods of diagnosing insulin resistance or a related condition in a mammal, by measuring Lcn2 activity in a biological sample obtained from the mammal, wherein an increase in Lcn2 activity is indicative of insulin resistance or related conditions. The invention further relates to use of compounds that reduce the activity or expression of Lcn2 for the manufacture of medicaments for reducing insulin resistance or increasing insulin sensitivity.

Using Lcn2 as a marker for insulin resistance or related conditions is advantageous because it does not require fasting or any special preparation by the patient, Lcn2 is a stable compound under routine collection conditions, and Lcn2 can be detected in a blood drop from a skin prick, or in urine. In addition, using Lcn2 as a marker for insulin resistance or related conditions may be useful in many at risk populations including obese and non-obese relatives of individuals with Type 2 diabetes patients with other criteria for the metabolic syndrome such as hypertension and in or hyperlipidemia and polycystic ovarian syndrome.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 demonstrates that Lcn2 is expressed in adipocytes and is regulated by Dex and TNF. Mature 3T3-L1 adipocytes were treated with Dex (1 μM) or TNF (4 ng/μL) in the presence or absence of rosiglitazone (Rosi; 1 μM), and Lcn2 mRNA levels were measured by Q-PCR. Data presented as mean±SD, *p<0.05, ***p=0.005 relative to no Rosi.

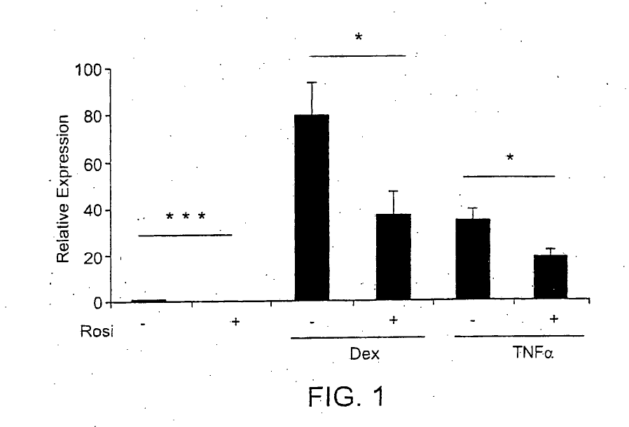

FIG. 2A-C depict the time course of Lcn2 mRNA expression during 3T3-L1 adipogenesis. Data are presented as mean±SD, n=3. FIG. 2A: Lcn2 mRNA expressiOn in confluent 3T3-L1 pre-adipocytes treated with Dex (D), MIX (M), Insulin (I) or combinations thereof. Data are presented as mean±SD, n=3. FIG. 1E: Lcn2 expression during 3T3-L1 differentiation induced by rosiglitazone, in the absence of DMI. Data are presented as mean±SD, n=3. For 2B and 2C, the inset shows the corresponding amount of Fabp4 mRNA to mark the extent of differentiation.

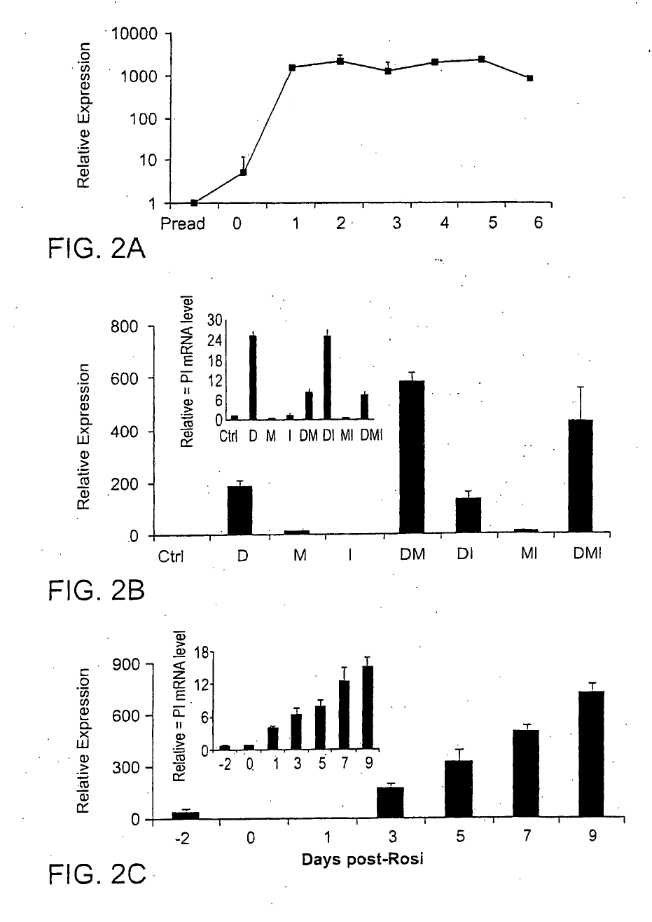

FIG. 3A-3D demonstrate Lcn2 expression in adipocytes is C/EBP dependent. FIG. 3A: PPAR γ−/− cells were infected with C/EBP-expressing retroviruses and endogenous levels of Lcn2 were measured by Q-PCR relative to cells transduced with empty vector. Data presented as mean±SD, *p<0.05, **p<0.01, ***p<0.001. FIG. 3B: Alignment of mouse (SEQ ID NO:19), rat (SEQ ID NO:20) and human (SEQ ID NO:21) Lcn2 promoter sequences reveals a putative C/EBP binding site. Boxed letters, core nucleotides essential for C/EBP binding. FIG. 3C: Deletion analysis of murine Lcn2 promoter fragments in transiently transfected NIH-3T3 cells in the presence (gray bars) or absence (white bars) of co-transfected C/EBPδ. Data represent mean±SD, n=3. FIG. 3D: Mutation analysis of the core C/EBP-binding motif. NIH-3T3 cells were transfected with the wild-type −222 fragment-luciferase construct or with the same fragment after mutation of the core TTGC in the presence (gray bars) or absence (white bars) of co-transfected C/EBPδ. Data represent mean±SD, n=6, # p=3.3e⁻¹⁰

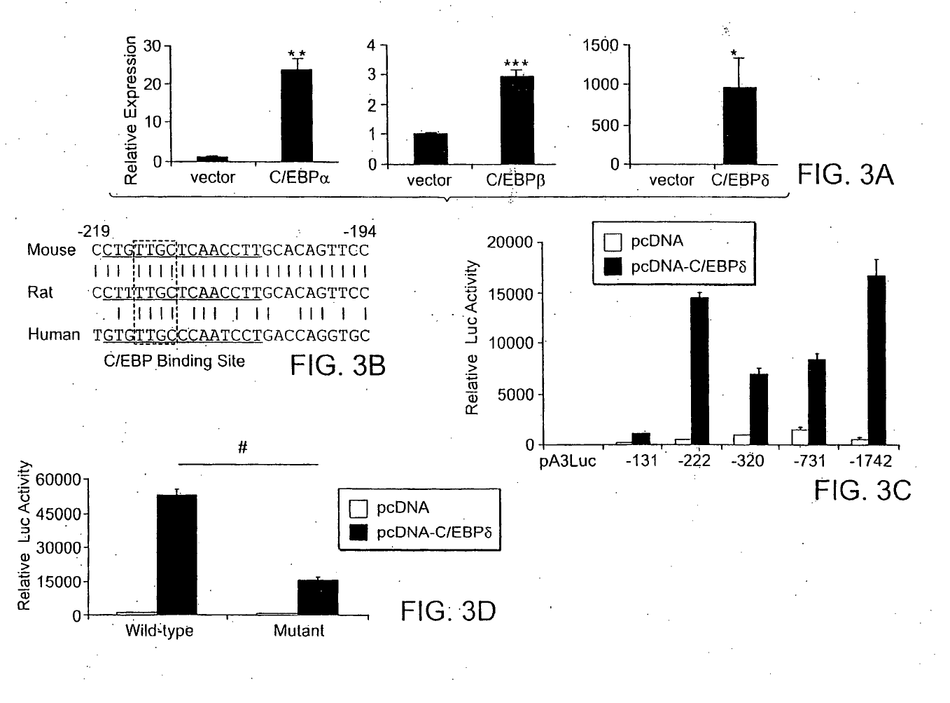

FIG. 4A-4E demonstrate that Lcn2 is elevated in obesity. FIG. 4A: Lcn2 protein levels in white adipose tissue lysates from ob/+ (n=5) and ob/ob (n=7) mice. Mean±SD, *p<0.05. FIG. 4B: Lcn2 mRNA expression in fractionated white adipose tissue from male C57BL mice given chow (n=7) or high-fat diet (n=7), relative to expression in chow macrophages. SVF=stromal vascular fraction, Ads=adipocytes, Macs=macrophages. Mean±SD, *p<0.05, ***p<0.001. FIG. 4C: Lcn2 protein levels in serum from fed ob/+ (n=6) and ob/ob mice (n=10), measured by Western blotting and expressed as fold relative to the mean of ob/+ controls. FIG. 4D: Lcn2 protein expression in serum from fed db/+ (n=8) and db/db mice (n=8), expressed as fold relative to the mean of db/+ controls. FIG. 4E: Lcn2 protein expression in serum from chow (n=15) and high-fat fed mice (n=18), expressed as fold relative to the mean of chow fed controls. Data for 4C, 4D, and 4E shown as the mean for each group.

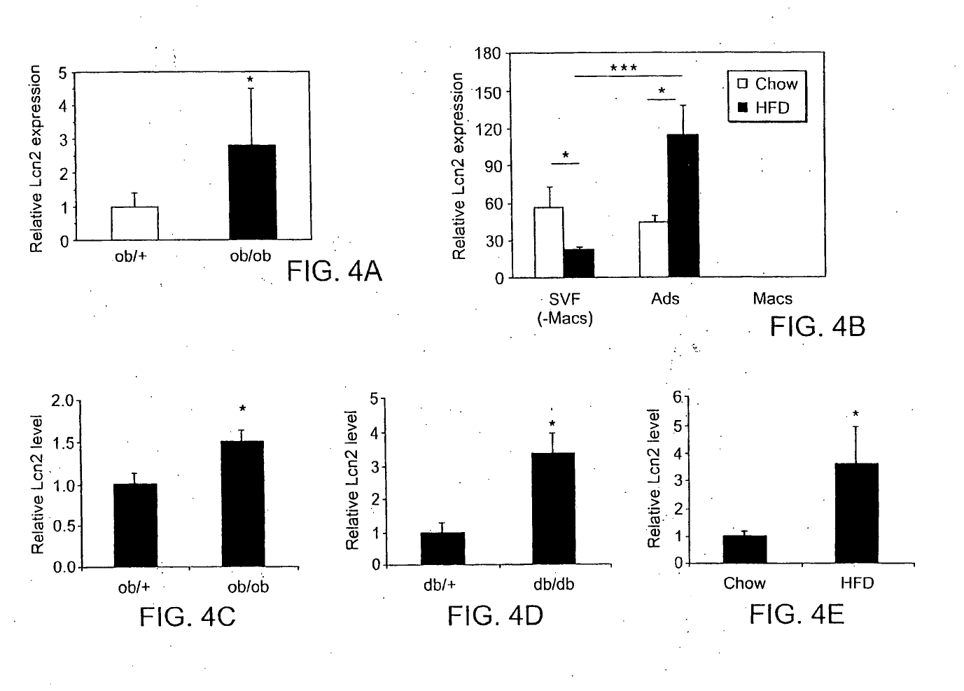

FIG. 5A-5C demonstrate that shRNA-mediated knockdown of Lcn2 improves insulin action. FIG. 5A: mRNA expression of Lon2 and markers in mature 3T3-L1 adipocytes expressing either control shRNA or shLcn2. FIG. 5B: Basal (white bars) and insulin-stimulated (black bars) glucose uptake in mature 3T3-L1 adipocytes expressing either control shRNA or shLcn2. n=12, mean±SD, *p<1e⁻⁵ relative to control shRNA, no insulin; # p=5e⁻⁵ relative to control shRNA, plus insulin. FIG. 5C: Component of glucose uptake attributable to insulin, equivalent to the uptake in the presence of insulin minus the uptake in the absence of insulin. n=12, mean±SD, *p<1e⁻⁴.

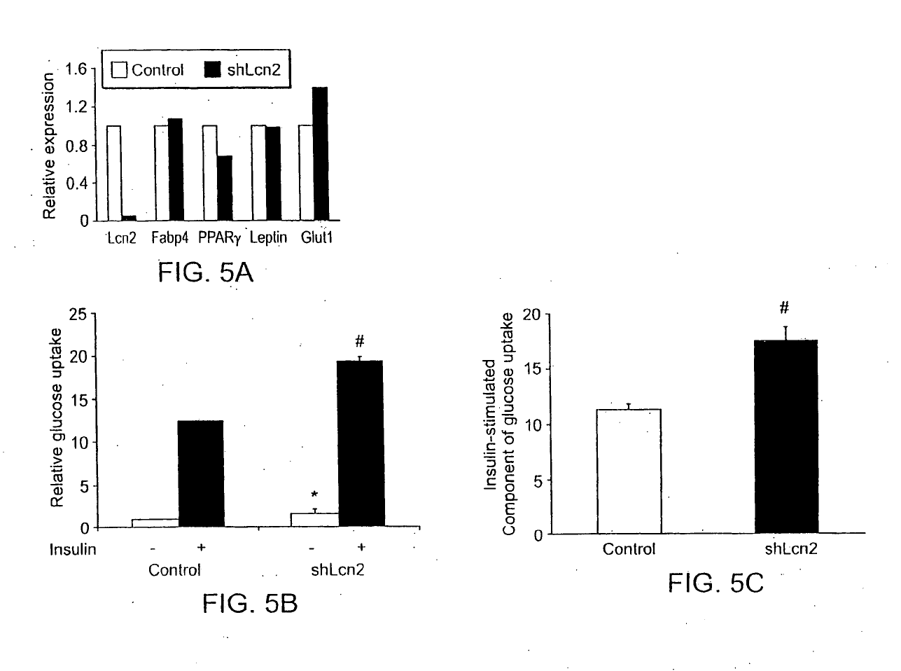

FIG. 6A-6C indicates that exogenous recombinant Lcn2 induces insulin resistance in H4IIe hepatocytes. FIG. 6A: Left, Glucose production induced by liganded Lcn2 (10 nM) or Dex (250 nM) in the presence or absence of insulin (100 nM). Right, effect of liganded Lcn2 (10 nM) or Dex (250 nM) on glucose-6-phosphatase mRNA expression in the presence or absence of insulin (100 nM). FIG. 6B: Dose response of liganded Lcn2 on glucose-6-phosphatase expression. FIG. 6C: Effect of apo-Lcn2 on glucose-6-phosphatase expression. For all panels, mean±SD, p<0.05, **p<0.01, n=3.

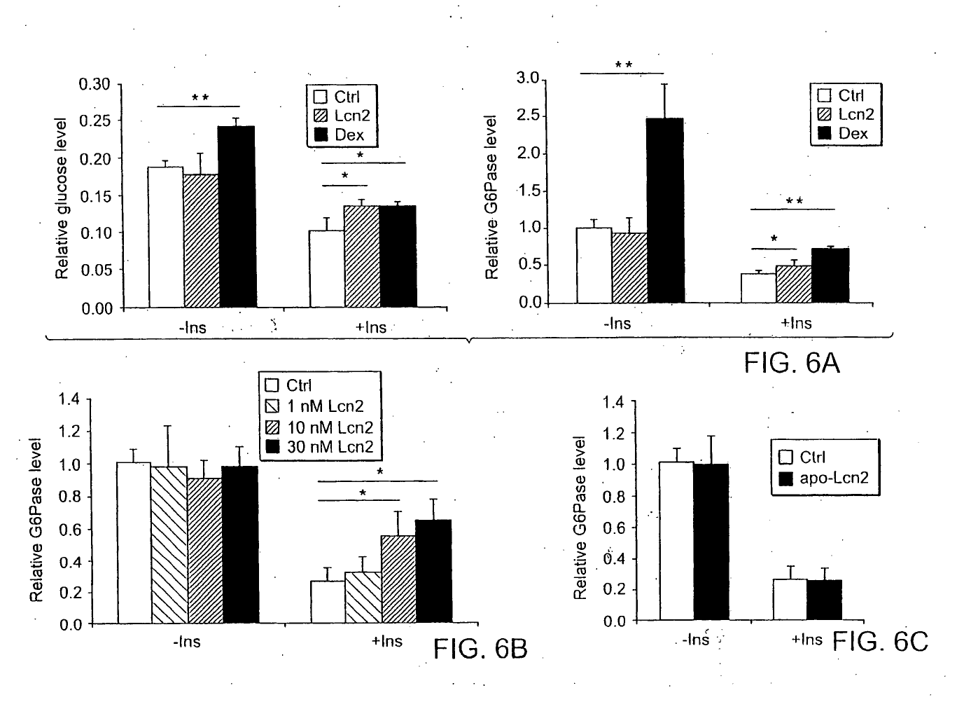

### DETAILED DESCRIPTION OF THE INVENTION

A description of example embodiments of the invention follows.

Applicant has identified lipocalina (Lcn2) as a factor dramatically induced by dexamethasone and by TNF-α in 3T3-L1 adipocytes. As described in detail below, Applicant has shown that adipose tissue is a dominant site of Lcn2 expression in the mouse, and that Lcn2 expression that it is regulated by obesity. In addition, it is demonstrated that Lcn2 promotes insulin resistance in adipocytes.

Methods for Screening Compounds that Modulate LCN2 Activity or Expression

As described herein, the present invention relates to methods for identifying compounds that modulate the activity or expression of Lcn2, either in vitro or in vivo (e.g., in a mammal), wherein the ability of the compound to modulate Lcn2 activity or expression was previously unknown. The methods of identification include in vitro or in vivo methods, and can be used to identify compounds that decrease Lcn2 activity or expression, or to identify compounds that increase Lcn2 activity or expression.

In one embodiment of identifying compounds that modulate Lcn2 activity, a test sample comprising Lcn2 is contacted with one or more test compounds. The term “lest sample,” as used herein, refers to a sample that comprises Lcn2 and/or comprises nucleic acid encoding Lcn2; representative test samples include, for example, biological samples such as a suitable cell, tissue, serum, plasma, or urine; alternatively, the test sample can be a cell-free sample, for example, a cell lysate, or a buffer comprising Lcn2.

In the methods, the level of Lent activity in the test sample in the presence of the test compound is compared with the level of Lcn2 activity in the absence of the test compound, wherein a difference in the level of Lcn2 activity is indicative of a compound that modulates Lcn2 activity. As described herein, Lcn2 activity includes, for example, the ability of Lcn2 to deliver iron, ability of Lcn2 to bind to siderophores or to a siderophore-iron complex, stability (e.g. structural, or half-life) of Lcn2 in tissues or in circulation, and the ability of Lcn2 to induce insulin resistance. In another embodiment, compounds that modulate the activity of Lcn2 reduce the level of insulin resistance in a mammal. Symptoms of insulin resistance include, for example, impaired glucose tolerance, impaired insulin-stimulated glucose transport, impaired insulin signaling, increased levels of serum glucose, and/or increased levels of serum insulin. These indicators of insulin resistance can be measured using standard methods in the art including the methods described herein.

In another embodiment, compounds that modulate Lcn2 expression are identified. “Expression,” as used herein, refers to expression of Lcn2 mRNA or protein. Lcn2 expression can be measured by detecting the level of Lcn2 mRNA in cells or tissue. Techniques for detecting RNA levels are well known in the art and include reverse transcriptase PCR (RT-PCR), Northern blotting, and RNAse protection assays. In addition, the rate at which Lcn2 mRNA is transcribed can be determined using a Lcn2 promoter reporter assay or a nuclear run-off assay. See “Current Protocols in Molecular Biology” Vol. 1, Chapter 4, John Wiley & Sons, Inc. (1997). Quantitative real time RT-PCR can be employed to assess Lcn2 mRNA stability. See Howe et al., Clin Chem. (2003); Bustin S A, J Mol Endocrinol. 29(1):23-29 (2002). See also, for example, U.S. Pat. No. 6,544,790, the teachings of which are incorporated by reference. Lcn2 expression can also be measured by detecting the level or concentration of Lcn2 protein or a biologically active fragment thereof. For example, any method suitable for detecting protein/peptide levels in tissue or cells can be used, such as specific antibody binding (immunological or immunoreactive method, e.g., ELISA, RIA, nephlometry or Western blot) to detect the levels of Lcn2, or a biologically active fragment thereof, or a characteristic fragment thereof (i.e., a fragment that may not have all of the biological activity of the intact Lcn2 protein, but can be used to specifically identify the biologically active protein).

Suitable cells or tissues for use in the assays for compounds that modulate Lcn2 activity or expression as described herein include, for example, adipose, liver, and muscle. Alternatively, for example, methods described herein can compare the level of Lcn2 in the blood of an individual (human or other mammal) prior to and after the administration of a test compound. Blood samples include, for example, whole blood, plasma, or serum. Urine, stool, and other bodily fluids can also be used. The assays can also include Lcn2 promoter-reporter assays, in vitro mRNA translation and stability assays, Lcn2 secretion assays using primary hepatocytes, or half-life studies of Lcn2 stability in cell culture conditions (ex-vivo) or in vitro. All of the assays described herein include high throughput assays.

Methods of identifying compounds that modulate Lcn2 activity also include in vivo methods. For example, the animal models for insulin resistance described herein can be used. In vivo methods of testing Lcn2 activity and/or insulin resistance include, for example, mice having insulin resistance such as AG4KO mice can be treated with or without the test compound and then subjected to glucose tolerance test or insulin tolerance test, wherein improved glucose tolerance or insulin tolerance is indicative of a compound that modulates Lcn2 activity. In another embodiment, the level of Lcn2 in serum can be compared between the two groups of mice, wherein the reduction in level of Lcn2 in the blood is indicative of a compound that modulates Lcn2 activity. Furthermore, blood glucose and plasma insulin level can be measured in mice, wherein a lower level of blood glucose and/or a lower plasma insulin level in treated mice compared to nontreated mice is indicative of a compound that modulates Lcn2 activity. In another embodiment, wild type mice can be administered a high-fat diet, and treated with the test compound, or not. The levels of Lcn2 can be compared between treated and nontreated mice wherein the reduction in the level of Lcn2, is indicative of a compound that modulates Lcn2 activity. Furthermore, the treated and nontreated mice on a high fat diet can be given the glucose tolerance test or the insulin tolerance test, wherein the reduction in glucose levels in the blood or plasma insulin levels is indicative of a compound that modulates or Lcn2 activity and thereby modulates insulin resistance. As used herein, modulation includes both inhibition and increase in activity, where inhibition is any measurable level of reduced activity, and increase is any measurable level of activity.

### Pharmaceutical Compositions

As described herein, a compound that modulates Lcn2 activity can also be useful for therapeutic treatment to alleviate conditions related to insulin resistance, as well as for the manufacture of medicaments for use in treatments to alleviate conditions related to insulin resistance. For example, the compounds can be used to reduce insulin resistance or increase insulin sensitivity.

Examples of the molecules that modulate Lcn2 activity (e.g., molecules that interfere with the activity of Lcn2) include molecules that structurally mimic the natural ligands of Lcn2, such as siderophores. Antibodies, either polyclonal, monoclonal, or antibody fragments that specifically bind to Lcn2 can also be used to interfere with Lcn2 activity. The production Of such specific antibodies is well-known to those of skill in the art.

Additionally, pharmaceutically acceptable salts of the disclosed compounds are included in the present invention. For example, an acid salt of a compound containing an amine or other basic group can be obtained, by reacting the compound with a suitable organic or inorganic acid, such as hydrogen chloride, hydrogen bromide; acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid.

Salts of compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethypamine, tri-(2-hydroxyethyl)amine, procaine, dibenzylpiperidine, N-benzyl-β-phenethylamine, dehydroabietylamine, N,N′-bisdehydroabietylamine, glucamine; N-methylglucamine, collidine, quinine, quinoline, and basic amino acid such as lysine and arginine.

The present invention includes pharmaceutical formulations of the compounds described herein. Pharmaceutical formulations can be adapted for administration by any appropriate route, for example by the oral (including buccal or sublingual), rectal, nasal, topical (including buccal, sublingual or transferal), vaginal or parenteral (including subcutaneous, intramuscular, intravenous or intradermal) route. Such formulations can be prepared by any method known in the art of pharmacy, for example by bringing into association the active ingredient with the carrier(s), diluent(s) or excipient(s).

Pharmaceutical formulations may be presented in unit dose forms containing a predetermined amount of active ingredient per unit dose. Such a unit may contain for example about 1 μg to 10 μg, about 0.01 mg to 1000 mg, or about 0.1 mg to 250 mg of the active ingredient, depending on the condition being treated, the route of administration and the age, weight and condition of the patient. In one embodiment; a retinamide, retinyl, or mimic thereof is administered orally, at a dose of about 10 to about 100 mg/day, or about 100 to about 500 mg/day or about 500 to about 1000 mg/day.

Pharmaceutical formulations adapted for oral administration may be presented as discrete units such as capsules or tablets; powders or granules; solutions or suspensions in aqueous or non-aqueous liquids; edible foams or whips; or oil-in-water-liquid emulsions or water-in-oil liquid emulsions.

Pharmaceutical formulations adapted for transferal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for parenteral administration include aqueous and non-aqueous sterile injection solutions which may contain the antioxidants as well as buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Suitable pharmaceutical carriers or diluents are typically inert ingredients that do not significantly interact with the active components of a pharmaceutical composition. The carriers or diluents should be biocompatible, i.e., non-toxic, non-inflammatory; non-immunogenic and devoid of other undesired reactions at the administration site. One of ordinary skill in the art is readily able to, select a carrier or diluent that is suitable for a particular method of administration or for a particular type of pharmaceutical composition (e.g., one containing retinamide or retinyl ester). Examples of pharmaceutically acceptable carriers and diluents include, for example, sterile water, physiological saline, bacteriostatic saline (saline containing about 0.9 mg/ml benzyl alcohol), phosphate-buffered saline, Hank's solution, Ringer's lactate, commercially available inert gels, or liquids supplemented with albumin, methyl cellulose or a collagen matrix. Additional carriers and diluents include sugars such as lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added (e.g., to a tablet or capsule), such as the cross-linked polyvinyl pyrrolidone, agar; or alginic acid or a salt thereof such as sodium alginate. Other carriers and diluents are described in Remington's Pharmaceutical Sciences, Mack Publishing Company, Easton; PA, the contents of which are incorporated by reference.

Pharmaceutical compositions of the invention can be prepared by combining, for example a retinamide or retinyl ester disclosed herein and a pharmaceutically active agent, and optionally including one of the carriers of diluents described above.

Also included in the present invention are pharmaceutically acceptable salts of the disclosed compounds. Depending on the charge of the compound, a salt will contain a positive ion or negative ion as a counterion. Compounds that have both a phosphate group and an amine group are considered to have no excess charge'. In this case, phosphate and amine groups can serve as counterions for each other or each group can have an exogenous counterion. Suitable cations include alkaline earth metal ions, such as sodium and potassium ions, alkaline earth ions, such as calcium and magnesium ions, and unsubstituted and substituted (primary, secondary, tertiary and quaternary) ammonium ions. Pharmaceutically acceptable counter anions include chloride, bromide, acetate, formate, citrate, ascorbate, sulfate and phosphate.

As used herein, the term “therapeutically effective amount” means the amount needed to achieve the desired therapeutic or diagnostic effect or efficacy. The actual effective amounts of the compound can vary according to the biological activity of the particular compound employed; specific drug or combination thereof being utilized; the particular composition formulated; the mode of administration the age, weight, and condition of the patient; the nature and severity of the symptoms or condition being treated; the frequency of treatment; the administration of other therapies; and the effect desired. Dosages for a particular patient can be determined by one of ordinary skill in the art using conventional considerations (e.g. by means of an appropriate, conventional pharmacological protocol).

For general information concerning formulations, see e.g., Gilman, et al. (eds.), 1990, Goodman and Gilman's: The Pharmacological Basis of Therapeutics, 8^{th} ed., Pergamon Press; and Remington's Pharmaceutical Sciences, 17^{th} ed., 1990, Mack Publishing Co., Easton, Pa.; Avis, et al. (eds.), 1993, Pharmaceutical Dosage Forms: Parenteral Medications, Dekker, New York; Lieberinan, eral. (eds.), 1990, Pharmaceutical Dosage Forms: Disperse Systems, Dekker, New York. The compounds of the present invention can be administered in conventional pharmaceutical administration forms, for example, uncoated or (film-)coated tablets, capsules, powders, granules, suppositories, suspensions or solutions. These are produced in a conventional manner. The active substances can for this purpose be processed with conventional pharmaceutical aids such as tablet binders, fillers, preservatives, tablet disintegrants, flow regulators, plasticizers, wetting agents, dispersants, emulsifiers, solvents, sustained release compositions, and/or antioxidants (cf. H. Sücker, et al.: Pharmazeutische Technologie, Thieme-Verlag, Stuttgart, 1978). The administration forms obtained in this way typically contain from about 1 to about 90 percent by weight of the active substance.

Methods of Reducing Insulin Resistance in an Individual

What is claimed is:

1. A method for identifying a compound that modulates Lcn2 activity, comprising contacting a test sample comprising Lcn2 with a test compound and comparing the level of Lcn2 activity in the presence of the test compound to the level of Lcn2 activity in the absence of the test compound to determine modulation of Lcn2 activity, wherein an alteration of Lcn2 activity is indicative of a compound that modulates Lcn2 activity.

2. The method of claim 1 wherein the level of Lcn2 activity in the presence of the test compound is reduced in comparison to the level of Lcn2 activity in the absence of the test compound.

3. The method of claim 1 wherein the test sample comprises cells.

4. A method for identifying a compound that modulates Lcn2 expression comprising contacting a test sample comprising cells expressing Lcn2 with a test compound and comparing the level of Lcn2 expression in the presence of the test compound to the level of Lcn2 expression in the absence of the test compound to determine modulation of Lcn2 expression, wherein an alteration of Lcn2 expression is indicative of a compound that modulates Lcn2 expression.

5. The method of claim 4 wherein the level of Lcn2 expression in the presence of the test compound is reduced in comparison to the level of Lcn2 expression in the absence of the test compound.

6. The method of claim 5 wherein the Lcn2 expression is expression of Lcn2 mRNA.

7. A method of reducing insulin resistance in a mammal, comprising administering to a mammal a compound that reduces Lcn2 activity.

8. A method of reducing insulin resistance in a mammal, comprising administering to a mammal a compound that reduces Lcn2 expression.

9. The method of claim 8 wherein the Lcn2 expression is expression of Lcn2 mRNA.

10. A method of increasing insulin sensitivity in a mammal, comprising administering to a mammal a compound that reduces Lcn2 activity.

11. A method of increasing insulin sensitivity in a mammal, comprising administering to a mammal a compound that reduces Lcn2 expression.

12. The method of claim 11 wherein the Lcn2 expression is expression of Lcn2 mRNA.

13. A method of diagnosing insulin resistance or a related condition in a mammal comprising measuring Lcn2 activity in a biological sample obtained from a mammal, wherein an increase in Lcn2 activity is indicative of insulin resistance or a related condition.

14. The method of claim 13 wherein the biological sample is selected from the group consisting of: tissue, whole blood, serum, plasma or urine.

15. The method of claim 14 wherein the amount of Lcn2 protein, or a fragment thereof, is measured.

16. The method of claim 15 wherein the amount of Lcn2 protein, or fragment thereof, is measured by immunoassay.

17. Use of a compound that reduces Lcn2 activity for the manufacture of a medicament for reducing insulin resistance in a mammal.

18. Use of a compound that reduces Lcn2 expression for the manufacture of a medicament for reducing insulin resistance in a mammal.

19. Use of a compound that reduces Lcn2 activity for the manufacture of a medicament for increasing insulin sensitivity in a mammal.

20. Use of a compound that reduces Lcn2 expression for the manufacture of a medicament for increasing insulin sensitivity in a mammal.
